(19) United States
(12) Patent Application Publication
(10) Pub. No.: US 2009/0191175 A1
(43) Pub. Date: Jul. 30, 2009

(54) THERAPEUTIC METHODS USING PEG-CONJUGATED AGENTS AND DEVICES FOR PERFORMING THE SAME

(75) Inventors: Jonathan K. ARMSTRONG, Los Angeles, CA (US); Timothy C. Fisher, La Crescenta, CA (US)

Correspondence Address: HOGAN & HARTSON L.L.P., 1999 AVENUE OF THE STARS, SUITE 1400, LOS ANGELES, CA, 90067, US

(73) Assignee: University of Southern California, Los Angeles, CA (US)

(21) Appl. No.: 12/390,032

(22) Filed: Feb. 20, 2009

Related U.S. Application Data
(63) Continuation-in-part of application No. 11/943,532, filed on Nov. 20, 2007.
(60) Provisional application No. 60/866,756, filed on Nov. 21, 2006.

(51) Int. Cl. A61K 35/12; A61K 38/46; A61M 1/34

(52) U.S. Cl. 424/93.73; 424/94.6; 604/5.01

(57) ABSTRACT

The present invention discloses methods and devices for selectively removing anti-PEG antibodies from a patient's blood prior to administration of a PEG-conjugated therapeutic agent so as to prevent immune reactions against the PEG-conjugated therapeutic agents when the agents are administered as a therapeutic regimen. Removal of anti-PEG antibodies may be physical, in which anti-PEG antibodies are physically removed from the blood through an extracorporeal blood circuit connected to an anti-PEG antibody removal device, or functional, in which an anti-PEG inactivating agent is infused into the patient's blood stream prior to administration of the PEG-conjugated therapeutic agent. Also disclosed is a device for selectively removing anti-PEG antibodies from a blood stream and system incorporating the device.

### CROSS-REFERENCE TO RELATED APPLICATIONS

This application is a continuation in part application of U.S. application Ser. No. 11/943,532, filed Nov. 20, 2007, which claims priority to Provisional Application No. 60/866,756 filed Nov. 21, 2006, entitled “POLY(ETHYLENE GLYCOL) ANTI-BODY DETECTION KIT”. The contents of the above mentioned applications are each expressly incorporated herein by reference.

### STATEMENT REGARDING FEDERALLY SPONSORED RESEARCH OR DEVELOPMENT

The present invention is made, at least in part, with the support of a grant from National Institute of Health, grant number HL65637. The government has certain rights in the invention.

### FIELD OF THE INVENTION

The present invention relates generally to methods of treating a patient with PEG-conjugated therapeutic agent. More particularly, the present invention provides a method that prevents immune reactions while treating patients with PEG-conjugated therapeutic agents.

### BACKGROUND OF THE INVENTION

There are many hormones, enzymes and other proteins that are, or could be, very useful for the treatment of diseases, but which are also highly immunogenic. If administered to humans, such substances may elicit a severe hypersensitivity reaction and/or be cleared very rapidly from the body by the immune system. One generally accepted approach for circumventing these limiting effects is to covalently conjugate the drugs to poly(ethylene glycol) (PEG) so as to mask the drugs from the body's immune system. PEG is a nonionic, water soluble synthetic polymer that is widely used in the food, cosmetic and pharmaceutical industries. It was first introduced for commercial applications in the early 1950s. PEG is also known as poly(ethylene oxide) (PEO) or polyoxyethylene (POE). Linear PEGs are commercially available from a molecular mass of 200 g/mol to in excess of 10 million g/mol. Most commercially available linear PEGs are synthesized using a difunctional initiator (e.g., ethylene glycol) which yields a dihydroxy terminated PEG chain. Methoxy-PEGs (mPEG) are synthesized by the addition of ethylene oxide to a monofunctional initiator (e.g., methanol as the simplest example). Different geometries of PEGs are also widely available: (i) multi-armed PEGs, with 3-8 arms emanating from a central group; (ii) star PEGs with 10 or more arms emanating from a central core group; (iii) comb- or graft-PEGs with multiple pendant chains from a polymeric backbone; and (iv) branched PEGs, formerly referred to as Y— or U-PEGs, have a central moiety to facilitate subsequent modification for attachment to a therapeutic agent. PEG is a nontoxic compound, and has been long believed to be non-immunogenic and non-antigenic. These properties are ideal for masking other substances with less than ideal immunogenic profiles. The development of PEG-conjugation has greatly extended the residence time in the circulation and greatly improved the efficacy of numerous therapeutically-useful proteins and enzymes. Some examples of PEG-conjugated therapeutic agents currently in clinical use include PEG-interferon, PEG-adenosine deaminase, PEG-erythropoietin, PEG-filgrastin and PEG-asparaginase. In addition, PEG-conjugation of numerous other therapeutic agents are in clinical use or development. Such therapeutic agents include, but are not limited to, insulin, arginine deaminase, glutaminase, camptothecin, recombinant human growth hormone, uricase, anti-tumor necrosis factor, anti-vascular endothelial growth factor, anti-granulocyte-stimulating factor, anti-granulocyte-macrophage colony stimulating factor, thrombopoietin, anti-glutamic acid decarboxylase, anti-bacillus anthracis exotoxin, photosensitizer immunoconjugates, benzoporphyrin derivative-anti-epidemial growth factor receptor, superoxide dismutase, glucocerebrosidase, hemoglobin, and liposomes.

However, there is a growing body of evidence suggesting that PEG-conjugated therapeutics are ineffective for some patients due to a specific antibody directed against the PEG molecule (anti-PEG) which causes rapid clearance of the PEG-conjugate. Some patients may have already developed the specific antibodies prior to treatment with the PEG-conjugated therapeutic as a result of environmental exposure to PEG (pre-existing anti-PEG). Other patients that initially respond to a PEG-conjugated therapeutic may later develop an anti-PEG antibody as a result of exposure to the PEG-conjugated therapeutic.

In view of the above problems, there is a need for improved methods that can enhance the effectiveness of PEG-conjugated therapeutic agents.

### SUMMARY OF THE INVENTION

Accordingly, it is an object of the present invention to provide a method to reduce or prevent immune reactions against PEG in patients who are receiving treatments utilizing a PEG-conjugated therapeutic agent. It is also an object of the present invention to provide devices and apparatus for facilitating treatment methods utilizing PEG-conjugated therapeutic agents without triggering immune reactions.

As mentioned in the background, anti-PEG immune reactions substantially reduce the effectiveness of PEG-conjugated therapeutic agents. Knowing that the cause of the reduced effectiveness of PEG-conjugated therapeutic agents is the presence of anti-PEG antibodies, there are several possible approaches to address the problem. In one approach, patients may be pre-screened for the presence of anti-PEG antibodies, as described in applicant's co-pending application Ser. No. 11/943,532. This approach can identify those patients who do not have anti-PEG and are therefore likely to benefit from the PEG-conjugated therapeutic agent, but does not offer a satisfactory solution for those patients who test positive for anti-PEG antibodies. The implication of this approach is that the benefits of PEG-conjugated therapeutic agents will be unavailable to the sub-population of patients who are anti-PEG positive. In accordance with this approach, conventional wisdom dictates that only non-PEGylated therapies should be used for patients who test anti-PEG positive. This situation is unfortunate since development of new therapeutic agents or delivery vehicles is usually expensive and difficult. It has been shown that approximately 25% of healthy individuals may be anti-PEG positive, which means that about a quarter of the population are not presently able to benefit from the excellent properties of PEG-conjugated therapeutic agents.

Rather than develop an alternative therapeutic agent, an alternative approach is to remove antibodies from the patient's blood stream, as is commonly done in organ transplantation. However, current antibody removal methods are not capable of selectively removing anti-PEG antibodies. For example, Tyden et al. has recently reviewed current methods of antibody removal (Tyden et al. Current Techniques of Antibody Removal, Transplantation, 2007; 84:S27-S29, the entire content of which is incorporated herein by reference), in which a representative selection of antibody removal methods are discussed, including those employing an immunoadsorbent column to remove antibodies. While immunoadsorbent columns such as protein-A column are available for removal of components of the immune system including IgG, IgM and other immuno-complexes, devices for specifically removing anti-PEG antibodies are heretofore unknown. Although it could prevent immune reactions against PEG-conjugated therapeutic agents, wholesale removal of antibodies suffers from the disadvantages of a high risk of infection due to removal of antiviral and antibacterial IgG and IgM.

The present invention offers a new approach to allow anti-PEG positive patients access to the benefits of PEG-conjugated therapeutic agents by selectively removing anti-PEG antibodies through the use of methods and devices of the present invention.

In a preferred embodiment, methods of the present invention will include the steps of selectively removing anti-PEG antibodies from the patient prior to administering the PEG-conjugated therapeutic agent. Removal of the anti-PEG antibodies may be physical or functional. Physical removal of the antibody may be accomplished, for example, in an apheresis process by withdrawing blood from the patient into an extracorporeal circuit, passing the blood through an antibody-removing device that is operatively connected to the extracorporeal circuit, and then returning the processed blood back to the patient. When the blood is passed through the antibody removing device, anti-PEG antibodies are sequestered by the device and removed from the blood stream of the patient. Anti-PEG antibody-removing devices of the present invention generally include anti-PEG antibody discriminating elements capable of forming antibody-antigen complexes with the anti-PEG antibodies. In effect, the antibody removing device acts as a selective anti-PEG antibody filter to filter out the undesirable anti-PEG antibodies from the patient's blood stream. The filtered blood is then returned to the patient from the extracorporeal circuit.

Functional removal of anti-PEG antibodies may be achieved by introducing an anti-PEG inactivating agent to render the anti-PEG antibodies non-reactive. In one exemplary embodiment, anti-PEG inactivating agents are introduced to a patient prior to administering a PEG-conjugated therapeutic agent to the patient. The anti-PEG inactivating agents of the present invention generally comprise anti-PEG antibody discriminating elements. They do not contain any therapeutic agents, but bind to anti-PEG antibodies within the patient's blood stream to form complexes which can no longer react with the PEG-conjugated therapeutic agent. In this way, the anti-PEG antibodies are functionally sequestered from the immune system of the patient. Accordingly, a PEG-conjugated therapeutic agent may then be administered to the patient without being made ineffective by the anti-PEG antibodies and without the risk of a hypersensitivity reaction.

Anti-PEG antibody-removing devices in accordance with embodiments of the present invention generally include a body having a fluid inlet, a fluid outlet, a fluid passageway extending from the fluid inlet to the fluid outlet, and a plurality of antibody discriminating elements enclosed therein, wherein the antibody discriminating elements are capable of recognizing and sequestering anti-PEG antibodies. The antibody discriminating elements may either be free standing or immobilized within the fluid passageway. When the patient's blood is passed through the fluid passageway, the anti-PEG antibodies are sequestered by the antibody discriminating elements and removed from the blood exiting the fluid passageway. Devices of the present invention may be incorporated in an apheresis system.

The above-mentioned and other features of this invention and the manner of obtaining and using them will become more apparent, and will be best understood, by reference to the following description, taken in conjunction with the accompanying drawings. The drawings depict only typical embodiments of the invention and do not therefore limit its scope.

### BRIEF DESCRIPTION OF THE DRAWINGS

FIG. 1 shows the result of a serological test using PEG-coated red blood cells (PEG-RBCs). Sample details are 1P) Buffer (negative); 2P) weak positive (2+w); 3P) weak positive (1+−2+w), 4P) strong positive (4+); 5P) negative, plasma sample from 4P pre-incubated with TentaGel beads to remove anti-PEG, 6P) negative.

FIG. 2 shows the result of a gel test using PEG-coated RBCs. Sample details are Ctrl) anti-PEG negative control plasma samples, PEG) and PEG2) anti-PEG positive plasma samples.

FIG. 3 shows the result of an assay in accordance with embodiments of the present invention with flow cytometric outputs using 10 μm diameter spherical TentaGel-OH beads stained for bound immunoglobulins with fluorescent secondary antibodies.

FIG. 4 shows images of serology tube testing results for various PEG-RBC incubation conditions described in Table 1. Plasma samples (Sample 1=negative for anti-PEG, Samples 2 and 3=positive for anti-PEG).

FIG. 5 shows images of serology gel test results for various PEG-RBC incubation conditions described in Table 1. Plasma samples (Sample 1=negative for anti-PEG, Samples 2 and 3=positive for anti-PEG).

FIG. 6 shows serological identification of anti-PEG versus asparaginase activity for PEG-asparaginase treated acute lymphoblastic leukemia (ALL) patients. Anti-PEG positive patients (squares, n=9) show undetectable ASNase activity while anti-PEG negative patients (triangles, n=19) show a mean ASNase activity of 353 U/L.

FIG. 7 shows cytometric identification of IgM anti-PEG. Anti-PEG positive patients (squares, n=13) show negligible ASNase activity (mean=12 U/L). Anti-PEG negative patients (triangles, n=15) show a high ASNase activity (mean=438 U/L). Mean data are shown with open symbols±S.D.

FIG. 8 shows an exemplary schematic of a conventional extracorporeal blood circuit.

FIG. 9 shows an exemplary anti-PEG antibody removing device in accordance with embodiments of the present invention.

### DETAILED DESCRIPTION

As used herein, the term “anti-PEG” refers to any species of antibodies that exhibit specific affinity for a PEG or the PEG component of a PEG-conjugated therapeutic agent. Because there may be more than one epitope on a PEG or a PEG-conjugated therapeutic agent, there may be a range of antibodies that exhibit specific affinities of varying degrees. For the purpose of this invention, the term anti-PEG is used to broadly refer to all antibodies that exhibit specific affinity for PEGs or PEG-conjugates, and except where indicated otherwise, is not intended to distinguish among different species or subtypes of anti-PEG antibodies. Specificity of an anti-PEG antibody can be easily determined by standard methods known in the art. For example, comparison of antibody binding to PEG and a suitable control substrate maybe used. Alternatively competitive binding assays employing another known anti-PEG may also be suitably used.

As used herein, the phrase “anti-PEG antibody discriminating elements” refers to any substance that is capable of acting as an antigen to an anti-PEG antibody. In some preferred embodiments, the antibody discriminating elements are a synthetic agent formed by immobilizing PEGs or PEG containing copolymers on a non-cross reacting substrate (i.e. substrates that do not react with anti-PEG). Exemplary non-cross reacting substrates may include red blood cells, glass beads, liposomes, metallic particles, polyamine core molecules (e.g., polyethylenimine, non-therapeutic proteins), polystyrene/divinyl benzene other non-PEG polymeric particles, nitrocellulose, PVDF membrane. The PEG molecules preferably have molecular masses between 300 g/mol to 500,000 g/mol. Immobilization of PEG is preferably achieved by covalently attaching the PEG molecules to the surface of the substrate. That is, the exposed surface of the substrate such as the surface of the glass beads will become coated by the PEG. Antigen probes formed by covalently attaching PEGs to a non-cross reacting particle are also referred to herein as PEG-coated particles. The density of PEG coating can vary depending on the substrate and the size and homogeneity of the PEG molecules.

When the antibody discriminating elements are formed by covalently attaching PEG molecules to red blood cells, they are also referred to herein as PEG-RBC. Covalent attachment of PEG to the surface of RBCs can be readily achieved by incubation RBCs in an appropriate physiological buffer with a PEG that has been functionalized with a reactive moiety. The PEG used in PEG-RBC preferably have molecular masses ranging from 300 g/mol to 50,000 g/mol. In one preferred embodiment, PEGs having molecular mass of about 20,000 g/mol are used.

In addition to PEG-RBCs and PEG-coated particles, various PEG polymers in particle form may also be used as anti-PEG antibody discriminating elements of the present invention. These PEG polymers are refereed to herein as PEG particles. Exemplary PEG particles may include the PEG-particles commercially available under the tradename TentaGel-OH particles (Rapp Polymere GmbH, Tübingen, Germany) which are primarily composed of PEG, and are available in discrete sizes from 4 to 300 μm diameter. Preferably, the PEG particles have a diameter ranging from about 2 microns to about 300 microns.

Additionally, other types of particles can be used such as PEG-liposomes, PEG-polymersomes, or beads with PEG grafted onto the surface, such as can be prepared by incubation of a chemically reactive PEG-derivative with suitably functionalized polystyrene beads (e.g., amine-functionalized beads).

While the above description of antibody discriminating elements envisions the elements as free standing particles that may be suspended or mixed in a solution, this is not a requirement. A person skilled in the art will readily recognize that other forms of presenting the elements may also be used. For example, PEGs can be directly immobilized to a plastic substrate such as that of a Corning 96-well plate typically used in the art.

For the purpose of the present invention, the act of contacting a patient's blood with the antibody discriminating elements may be performed in a number of ways depending on the specific embodiment of the antibody removing device, so long as the blood come into physical contact with the antibody discriminating elements. In those embodiments where the antibody discriminating elements are free standing particles, contacting also preferably includes mixing.

In general, methods in accordance with embodiments of the present invention will include the steps of removing anti-PEG antibodies from the patient prior to administering a PEG-conjugated therapeutic agent. Removal of anti-PEG antibodies can be achieved through a number of different ways. In a preferred embodiment, an apheresis procedure is used to draw blood from the patient and passing it through an extracorporeal circuit before returning the blood back to the patient.

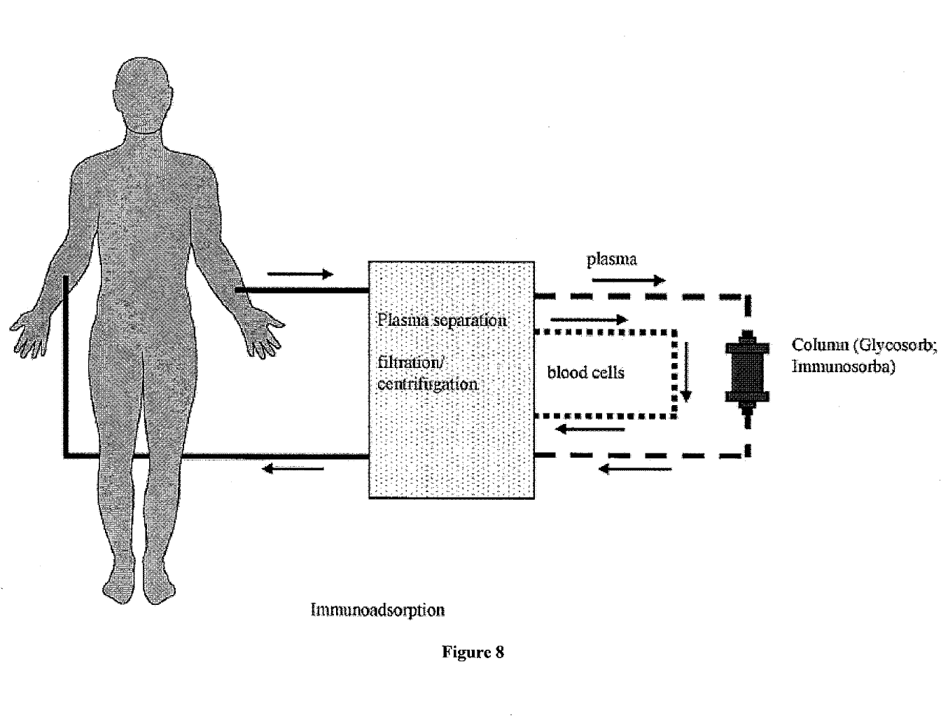

It will be appreciated by those skilled in the art that extracorporeal blood circuits have been extensively used in a number of different therapeutic protocols, such as in extracorporeal photodynamic therapy or in plasmapheresis for organ transplantation, and may be adapted for use in methods of the present invention. FIG. 8 shows a conventional extracorporeal circuit connected to a patient for plasmapheresis. In this circuit, blood is first separated into plasma and blood cells. The plasma is then passed through a protein-A immunoadsorbent column to remove antibodies and other immune complexes from the plasma. In exemplary embodiments of the present invention, specific removal of anti-PEG antibodies may be accomplished by substituting the protein-A column with an anti-PEG antibody removing device of the present invention.

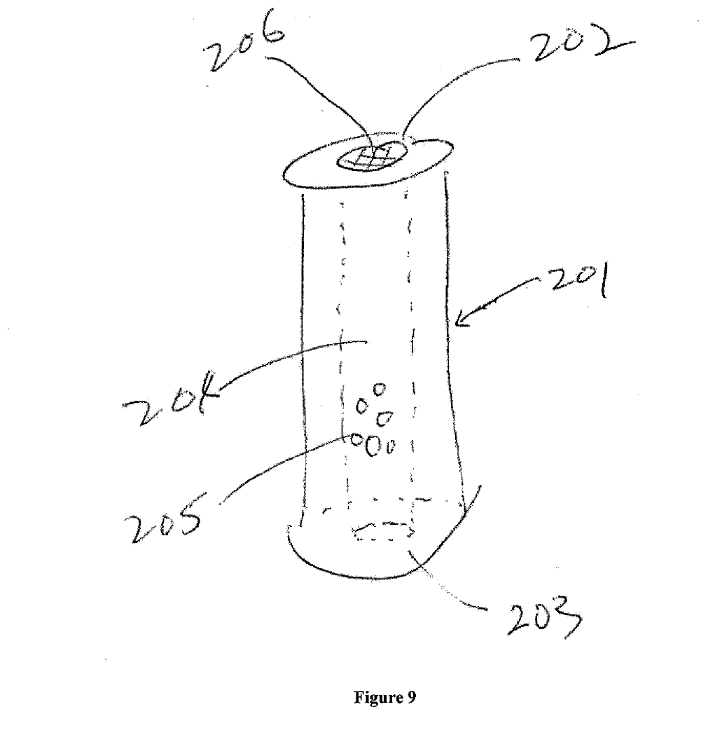

Anti-PEG antibody removing device in accordance with embodiments of the present invention are preferable in the form of an immunosorbent column. Referring to FIG. 9, in a preferred embodiment, an anti-PEG antibody removing device will have a tubular body 201, a fluid inlet 202, a fluid outlet 203, and a fluid passageway 204 extending from the fluid inlet 202 to the fluid outlet 203. A plurality of antibody discriminating elements 205 are contained within the passageway.

The antibody discriminating elements 205 may either be free standing or immobilized within the fluid passageway. In those embodiments where the antibody discriminating elements 205 are free standing, a barrier 206 is preferably present at the fluid inlet 202 and the fluid outlet 203 to prevent the antibody discriminating elements from exiting the fluid passageway.

In a further preferred embodiment, the anti-PEG antibody removing device may further comprise apparatus for incubating the blood with the anti-PEG antibody discriminating elements to facilitate antigen-antibody complex formation.

Since the anti-PEG antibody removing device is typically used together with an extracorporeal circuit, the present invention also provides a system for selectively removing anti-PEG antibodies from a patient which includes an extracorporeal blood circuit and an anti-PEG antibody removing device of the present invention operatively connected thereto.

In an alternative embodiment, the anti-PEG antibodies are functionally inactivated rather than physically removed from the patient. In this embodiment, anti-PEG antibody inactivating agents are infused into the patient's blood stream prior to or concurrently with the administration of a PEG-conjugated therapeutic.

In a further embodiment, a screening test may be performed to determine the type of anti-PEG antibodies present in the patient so as to more selectively choose the antibody inactivating agents to be administered.

Depending on the specific antigen probe-biological sample pair, suitable analysis known in the art may be selected. Exemplary techniques for detecting the antigen-antibody complex in a sample may include serological assay, fluorescent assay, enzyme-linked assay, flow cytometry, lateral flow assay, or any other techniques commonly known in the art. Details for methods of assaying anti-PEG antibodies and kits useful for performing such assays are fully described in applicant's co-pending application Ser. No. 11/943,532.

The following examples are intended to illustrate, but not to limit, the scope of the invention. While such examples are typical of those that might be used, other procedures known to those skilled in the art may alternatively be utilized. Indeed, those of ordinary skill in the art can readily envision and produce further embodiments, based on the teachings herein, without undue experimentation.

### EXAMPLES

### Example 1

### Testing for an Antibody to Poly(Ethylene Glycol)

### Method:

### Preparation of Antigen:

Blood type O red blood cells (RBCs) were washed 3 times with phosphate buffered saline (PBS, pH 7.4, 290 mOsm/kg) at 1400×g for 6 minutes. RBCs were then resuspended to a 10% hematocrit (hct) in 15 mM triethanolamine buffer (pH 8.4, 290 mOsm/kg). Poly(ethylene glycol) coating of RBCs was achieved by the addition of a reactive PEG to the RBC suspension. A succinimidyl propionate derivate of monomethoxy-poly(ethylene glycol) of molecular mass 20 kDa (mPEG20k-SPA) was dissolved in cold 10 mM hydrochloric acid+154 mM NaCl, and added to the RBC suspension to yield a suspension phase concentration of 5 mg/mL mPEG20k-SPA. The mixture was incubated at room temperature for 1 hour, and then washed 3 times with PBS at 500×g for 10 minutes. PEG-RBCs were then resuspended to a 5% hct in PBS and used for serologic testing.

### Tube Test for Anti-PEG:

One drop of RBCs (PEG-coated or control (uncoated) RBCs) at 5% hot were added to 2 drops of plasma. Samples were incubated at room temperature for 15 minutes and then centrifuged at 1000×g for 1 minute. Agglutination of PEG-coated or uncoated (control) RBCs by each plasma sample was scored according to the 0-4+ scale. An anti-PEG positive sample was identified as one where agglutination of PEG-RBCs was observed in the absence of agglutination of control (uncoated) RBCs. An anti-PEG negative sample was identified as one where no agglutination was observed with PEG-RBCs.

### Confirmation of Specificity of Anti-PEG:

To confirm specificity of anti-PEG following a positive serological test with PEG-RBCs, the test was repeated following removal of anti-PEG from the sample by pre-incubation with PEG-particles: Four hundred μL of anti-PEG positive plasma were added to 20 mg of 10 μm TentaGel-OH suspended in 200 μL of PBS and incubated at room temperature for 30 minutes to specifically adsorb any anti-PEG present. The mixture was then centrifuged at 1000×g for 5 minutes, and the supernatant separated for testing, Testing was repeated as described above with PEG-RBCs. Anti-PEG specificity was confirmed if the agglutination of PEG-RBCs was eliminated after adsorption with PEG.

### Results:

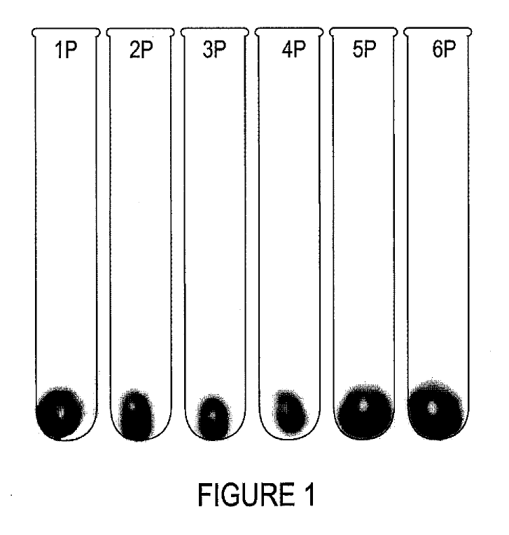

The results of serological testing with PEG-RBCs are shown in FIG. 1. No agglutination was observed with uncoated (control) RBCs (images not shown). The sample details for FIG. 1 are as follows: Sample 1P) shows PEG-RBCs in buffer (negative); Samples that are anti-PEG positive are 2P) weak positive (2+w); 3P) weak positive (1+-2+w) and 4P) strong positive (4+). The anti-PEG specificity was confirmed by adsorption of sample 4P with PEG-particles, resulting in an absence of agglutination (sample 5P). Sample 6P tested negative for anti-PEG (i.e., no agglutination of PEG-RBCs).

### Example 2

### Gel Test for an Antibody to Poly(Ethylene Glycol)

### Preparation of Antigen:

PEG-coated RBCs were prepared as described in Example 1.

### Preparation of Gel Test Tubes:

Gel Test tubes were prepared as follows. One hundred microliters of Sephacryl 500-HR beads at 50% solids were pipetted into a narrow 300 μL tube. The tube was centrifuged at 500×g for 10 minutes.

### Example of Gel Test for Anti-PEG:

One hundred microliters of plasma was then pipetted on the top of the gel layer. Twenty five microliters of PEG-RBCs (or control (uncoated) RBCs) at 10% hct were added to the top of the plasma layer. Samples were incubated at room temperature for 15 minutes, and then centrifuged at 500×g for 3 minutes

Agglutination was scored accordingly: RBCs do not enter top of gel=4+ positive test, RBCs pass through gel=O negative test.

### Results:

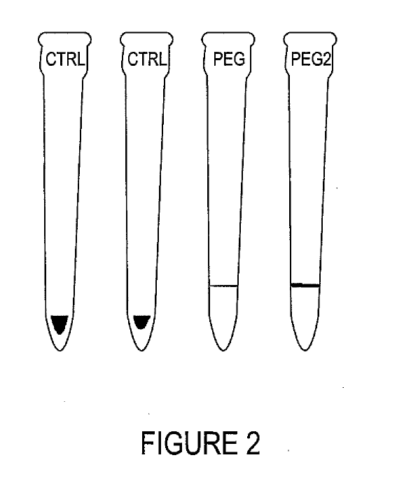

The results of the gel test with PEG-RBCs are shown in FIG. 2. All uncoated (control) RBCs passed through the gel and gave a negative gel test (images not shown). The sample details for FIG. 2 are as follows: Plasma samples labeled “Ctrl” are anti-PEG negative samples as PEG-RBCs passed through the gel giving a negative gel test. Plasma samples labeled “PEG” and “PEG2” are anti-PEG positive showing a strong 4+ agglutination test result.

### Example 3

### Flow Cytometric Testing for an Antibody to Poly(Ethylene Glycol)

### Method:

Fifty microliters of each test plasma were added to 100 μL of PBS and 25 μL of a 1% suspension of 10 μm diameter poly(ethylene glycol) particles, known commercially as TentaGel-OH beads (TentaGel-OH M 30 100, Rapp Polymere GmbH, Tübingen, Germany), which are composed primarily of PEG. The mixture was incubated for 1 hour at room temperature and the beads were washed twice with PBS (200×g for 2 minutes) and resuspended with 1 mL of PBS containing 5 μL of fluorescein isothiocyanate labeled-anti-human IgG and 5 μL of R-phycoerythrin labeled-anti-human IgM. After 1 hour incubation at room temperature in the dark, the particles were washed 3 times with PBS (200×g for 2 minutes) and resuspended with 0.5 mL of PBS and examined by flow cytometry. Ten thousand counts were recorded per sample, gated for single beads. Non-specific protein uptake was investigated by staining with FITC-anti-human albumin.

### Results:

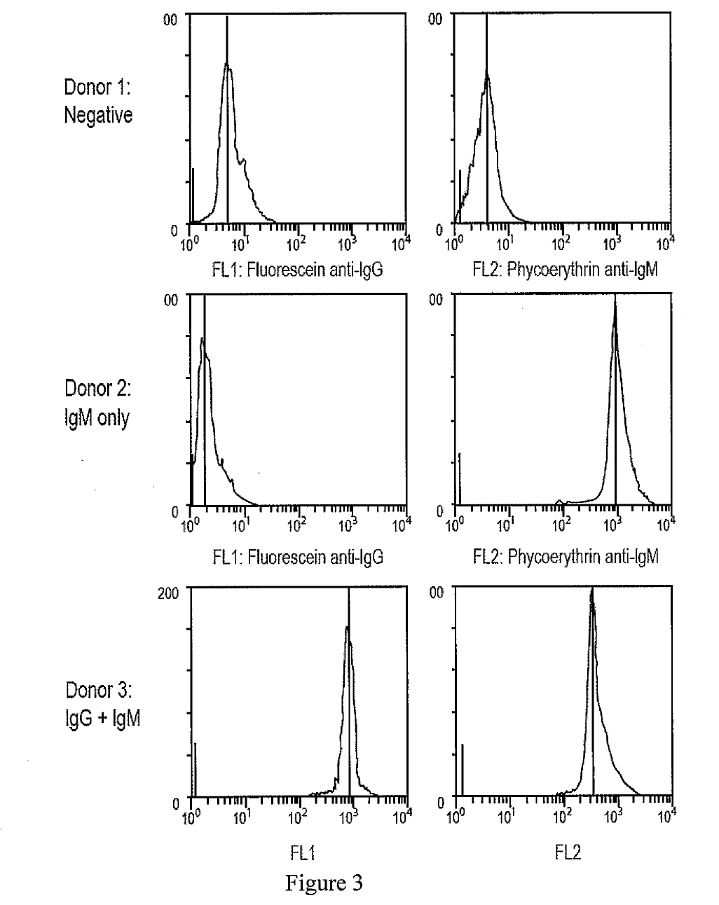

Representative data for human plasma samples are shown in FIG. 3. Donor 1 shows no evidence of bound antibody (anti-PEG negative), Donor 2 shows evidence of IgM binding only (anti-PEG positive, IgM), Donor 3 shows binding of both IgG and IgM (anti-PEG positive, IgG and IgM). No evidence of albumin binding to TentaGel-OH beads was observed for all sera tested.

### Example 4

### Variation of Reactive PEG Concentration

### Method:

Freshly drawn blood type O RBCs were PEG-coated according to the protocol described in Example 1, but with the following variations to demonstrate the effect of incubation hematocrit (hct) and reactive PEG concentration:

### Incubation at 50% hct:

Washed RBCs were resuspended in triethanolamine buffer at 50% hct. A succinimidyl propionate derivate of monomethoxy-poly(ethylene glycol) of molecular mass 20 kDa (mPEG20k-SPA) was dissolved in cold 10 mM hydrochloric acid+154 mM NaCl and added to RBC alquots to achieve the following concentrations:
5 mg/mL of suspension (10 mg/mL RBCs)
10 mg/mL of suspension (20 mg/mL RBCs)
20 mg/mL of suspension (40 mg/mL RBCs)

Washed RBCs were resuspended in triethanolamine buffer at 10% hct. A succinimidyl propionate derivate of monomethoxy-poly(ethylene glycol) of molecular mass 20 kDa (mPEG20k-SPA) was dissolved in cold 10 mM hydrochloric acid+154 mM NaCl and added to RBC alquots to achieve the following concentrations:
1 mg/mL of suspension
5 mg/mL of suspension
10 mg/mL of suspension

The mixtures were incubated at room temperature for 1 hour, and then washed 3 times with PBS at 500×g for 10 minutes. PEG-RBCs were then resuspended to a 5% hct in PBS and used for serologic testing. PEG-coated RBCs were evaluated with autologous plasma (Sample 1, anti-PEG negative) and two anti-PEG positive plasma samples (Samples 2 and 3) using tube and gel tests described in Example 1 and Example 2 respectively.

### Results:

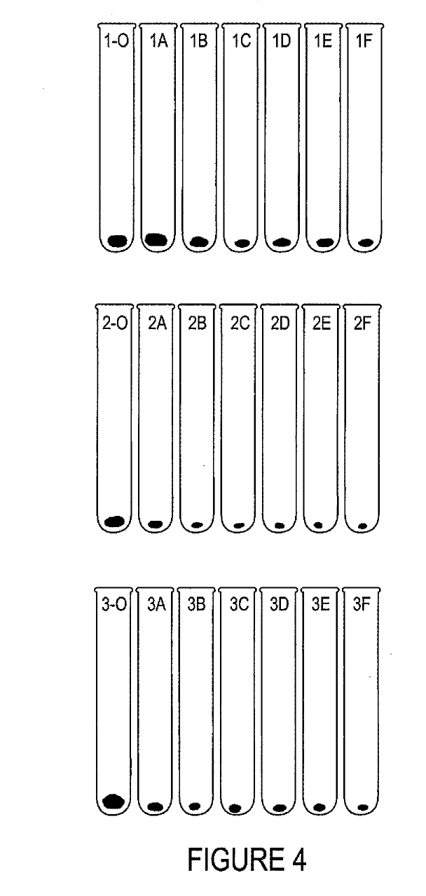

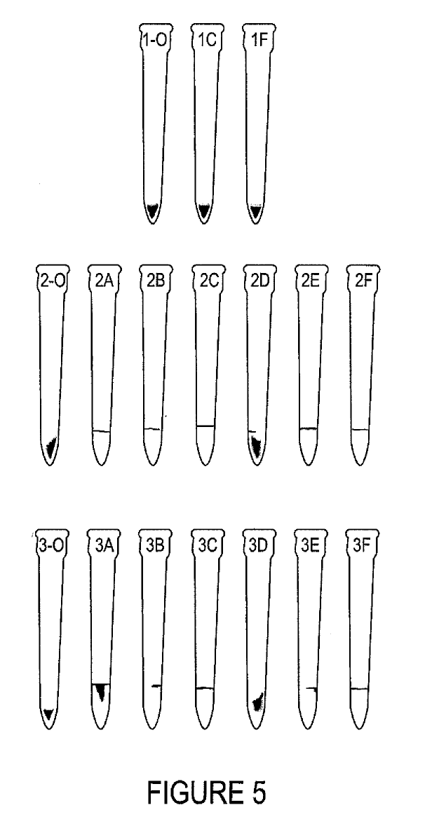

Table 1 below shows the tube and gel test results for PEG-RBCs (and control, uncoated RBCs) incubated with three plasma samples (Sample 1=negative for anti-PEG, Samples 2 and 3=positive for anti-PEG). Images of the tube and gel test samples are also shown in FIGS. 4 and 5.

TABLE 1
PEG-coating details, tube and gel test results for anti-PEG
with 3 plasma samples (Sample 1 = negative for anti-PEG,
Samples 2 and 3 = positive for anti-PEG).

| | Sample Label | | | | | | |
|---|---|---|---|---|---|---|---|
| | 0 | | | | | | |
| Details | (Ctrl) | A | B | C | D | E | F |
| Incubation hct | N/A | 50% | | | 10% | | |
| Reactive PEG | 0 | 5 | 10 | 20 | 1 | 5 | 10 |
| Conc (mg/mL) | | | | | | | |

Tube Test

| | | | | | | | |
|---|---|---|---|---|---|---|---|
| Sample 1 (Neg) | 0 | 0 | 0 | 0 | 0 | 0 | 0 |
| Sample 2 (pos) | 0 | 4+ | 4+ | 4+ | 2+/3+ | 4+ | 4+ |
| Sample 3 (pos) | 0 | 3+ | 3+ | 3+ | 2 + w | 3+ | 4+ |

Gel Test

| | | | | | | | |
|---|---|---|---|---|---|---|---|
| Sample 1 (neg) | 0 | | | 0 | | | 0 |
| Sample 2 (pos) | 0 | 4+ | 4+ | 4+ | 1+ | 4+ | 4+ |
| Sample 3 (pos) | 0 | 2+ | 4+ | 4+ | 0 | 3+ | 4+ |

### Conclusions:

Preparation of Reagent PEG-RBCs for Screening Plasma Samples for the Presence of Anti-PEG may be performed using an incubation hematocrit of 10-50% and a reactive PEG concentration of 5 to 10 mg/mL at 10% hct, and 10 to 20 mg/mL at 50% hct.

### Example 5

### Identification of Anti-PEG Epitope

PEG-RBCs were prepared and serological testing performed as described in Example 1. Three anti-PEG positive sera that gave a strong agglutination (4+) result with PEG-RBCs using the tube test were used for the anti-PEG epitope study. Two percent (w/v) solutions of various polymers [PEG of molecular mass 300 g/mol and 20,000 g/mol, monomethoxy-PEG of molecular mass 5000 g/mol, dextran of molecular mass 40,000 g/mol, polyvinylalcohol (PvOH) of molecular mass 25,000 g/mol, poly(propylene glycol) (PPG) of molecular mass 2000 g/mol] and small ethers and ether oligomers [di- to penta-(ethylene glycol); di- to tetra-(ethylene glycol) dimethyl ether] were prepared in PBS. The polymer and small ether solutions were added to anti-PEG positive sera at a 1:1 (v/v) ratio giving a final polymer/small ether concentration of 1% (w/v) and incubated for 30 minutes at room temperature. Agglutination testing with PEG-RBCs was then performed as described in Example 1. Inhibition of agglutination by the smallest ether molecule tested determined the anti-PEG epitope.

### Results:

Complete inhibition of agglutination (from 4+ to 0) was observed in the presence of all PEGs (MW 300-20,000 g/mol), PPG (2000 g/mol), tri- and tetra-(ethylene glycol)dimethyl ether and penta(ethylene glycol). Di(ethylene glycoldiethyl ether and tetra(ethylene glycol) reduced PEG-RBC agglutination (4+ to 2+). Dextran, PvOH, ethylene glycol, di- and tri-(ethylene glycol) had no effect.

### Conclusion:

Comparison of the smallest inhibitors indicates that the minimum epitope required for binding of the PEG-antibody is a backbone of 4 to 5 repeat —(C—O—C)— units.

TABLE 2
Agglutination of PEG-RBCs by 3 normal plasma samples (4+ reactive for anti-PEG) in presence of 1%
w/v PEG, PEG oligomers and analogs, PPG, and irrelevant polymers (dextran 40 and polyvinyl alcohol).

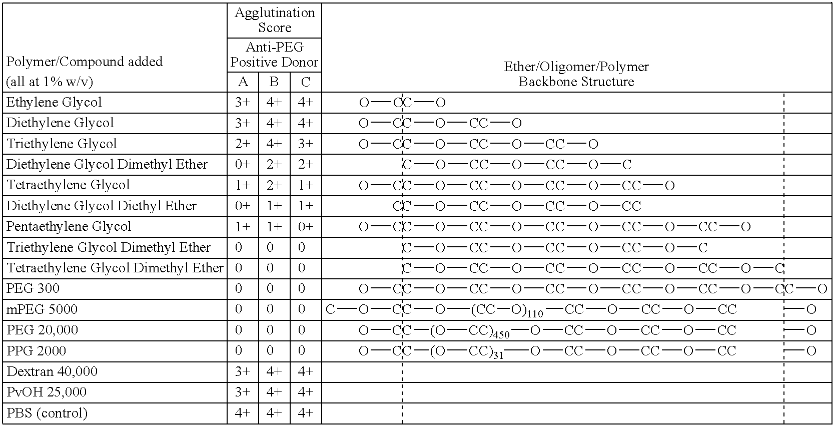

### Example 6

### Serology Screening of Healthy Blood Donors for Anti-PEG

Plasma samples were collected from 350 normal healthy subjects. One drop of RBCs (PEG-coated or control (uncoated) RBCs) at 5% hct were added to 2 drops of plasma. Samples were incubated at room temperature for 15 minutes and then centrifuged at 500×g for 1 minute. Agglutination of PEG-coated or uncoated (control) RBCs by each plasma sample was determined using the serological tube test, and agglutination scored according to the 0-4+ scale

### Results:

94 plasma samples (26.9%) agglutinated PEG-RBCs (26.9% positive for anti-PEG), 74 of these (21.2%) scored 1+ to 2+, and 20 (5.7%) showed strong agglutination (3+ or 4+) (Table 3). No agglutination was observed for control (uncoated) RBCs in patient sera.

TABLE 3
Testing of normal donor sera with PEG-coated
RBCs scored according to the 0-4+ scale.

| Agglutination score | Number | Percentage |
|---|---|---|
| 0 | 256 | 73.1 |
| 1+ | 31 | 8.9 |
| 2 + w | 26 | 7.4 |
| 2+ | 17 | 4.9 |
| 3+ | 12 | 3.4 |
| 4+ | 8 | 2.3 |
| Negative | 256 | 73.1 |
| Positive | 94 | 26.9 |
| Total | 350 | 100 |

### Example 7

### Flow Cytometric Screening of Healthy Blood Donors for Anti-PEG and Identification of IgG Sub-Types

### Method:

Fifty microliters of test plasma were added to 100 μL of PBS and 25 μL of a 1% suspension of 10 μm diameter poly(ethylene glycol) particles, known commercially as TentaGel-OH beads (TentaGel-OH M 30 100, Rapp Polymere GmbH, Tübingen, Germany), which are composed primarily of PEG. The mixture was incubated for 1 hour at room temperature and the beads were washed, twice with PBS (200×g for 2 minutes) and resuspended with 1 mL of PBS containing 5 μL of fluorescein isothiocyanate labeled-anti-human IgG and 5 μL of R-phycoerythrin labeled-anti-human IgM. After 1 hour incubation at room temperature in the dark, the particles were washed 3 times with PBS (200×g for 2 minutes) and resuspended with 0.5 mL of PBS and examined by flow cytometry. Ten thousand counts were recorded per sample, gated for single beads.

### Testing for IgG Sub-Types:

Twelve anti-PEG positive sera, and one anti-PEG negative serum were also examined for IgG sub-types. Sera were incubated with 10 μm diameter TentaGel-OH beads as described above. Each sample was divided into 4 equal aliquots and stained for bound IgG sub-types with 1 mL of PBS containing 5 μL of fluorescein isothiocyanate labeled-anti-human IgG-1, IgG-2, IgG-3 or IgG-4. Flow cytometric analyses were performed as described above.

### Results:

Flow cytometric analysis of TentaGel-OH beads showed 97 samples (27.7%) positive for IgG and/or IgM (27.7% positive for anti-PEG), of which 67 samples (19.1%) showed IgG binding only, 18 (5.1%) showed IgM only, and 12 (3.4%) showed both IgG and IgM uptake (Table 4). No evidence of albumin uptake was observed, which argues against non-specific protein binding. Analysis of 12 anti-PEG positive sera for IgG subtypes showed that of 11 sera that were positive of anti-PEG IgG, all 11 were positive for anti-PEG IgG-2, and one sera sample was positive for IgG-1, IgG-2 and IgG-3 (Table 5). One anti-PEG positive sera that was anti-PEG IgM only (sample labeled “V” in Table 5) tested negative for all IgG subtypes analyzed. No anti-PEG positive sample showed evidence of IgG-4.

TABLE 4
Flow cytometric analysis of TentaGel-OH beads incubated
in normal donor sera and stained for bound IgG and IgM.

| | Number | Percentage |
|---|---|---|
| IgG only | 67 | 19.1 |
| IgM only | 18 | 5.1 |
| IgG + IgM | 12 | 3.4 |
| Negative | 253 | 72.3 |
| Positive | 97 | 27.7 |
| Total | 350 | 100 |

Mean fluorescence data were employed to identify the presence or absence of anti-PEG in test sera.

TABLE 5
Flow cytometric analysis of TentaGel-OH beads incubated
in normal donor anti-PEG positive sera and stained for
bound IgG sub-types (IgG-1, -2, -3 and -4).

| | Mean Fluorescence Intensity of | | | | |
|---|---|---|---|---|---|
| Donor | TentaGel-OH beads | | | | Anti-PEG |
| # | IgG-1 | IgG-2 | IgG-3 | IgG-4 | Sub-Type |
| P | 7.3 | 4.9 | 17 | 0.6 | IgG-1, -2 and -3 |
| T | 0.4 | 6.1 | 0.4 | 0.4 | IgG-2 |
| 219 | 0.4 | 1.5 | 0.5 | 0.4 | IgG-2 |
| 238 | 0.5 | 1.4 | 0.5 | 0.4 | IgG-2 |
| 243 | 0.5 | 1.1 | 0.4 | 0.3 | IgG-2 |
| 253 | 0.4 | 2 | 0.3 | 0.3 | IgG-2 |
| 267 | 0.6 | 11.4 | 0.5 | 0.4 | IgG-2 |
| 282 | 0.6 | 1.7 | 0.5 | 0.4 | IgG-2 |
| 292 | 0.5 | 4.6 | 0.6 | 0.3 | IgG-2 |
| 294 | 0.6 | 2.1 | 0.6 | 0.5 | IgG-2 |
| 299 | 0.4 | 5.8 | 0.4 | 0.3 | IgG-2 |
| V | 0.3 | 0.3 | 0.4 | 0.3 | (IgM only) |
| J | 0.3 | 0.2 | 0.4 | 0.3 | Negative Control |

Mean fluorescence data for each analysis is given, with a positive result given in bold text.

### Example 8

### Testing of Patient Sera for Anti-PEG

### Methods:

Stored serum samples were collected from 28 pediatric acute lymphoblastic leukemia (ALL) patients who received the chemotherapy agent, PEG-conjugated asparaginase (PEG-ASNase). Sera were assayed for ASNase activity using a microplate technique. Testing for anti-PEG was performed by serology as described in Example 1 and by flow cytometry as described in Example 3.

### Results:

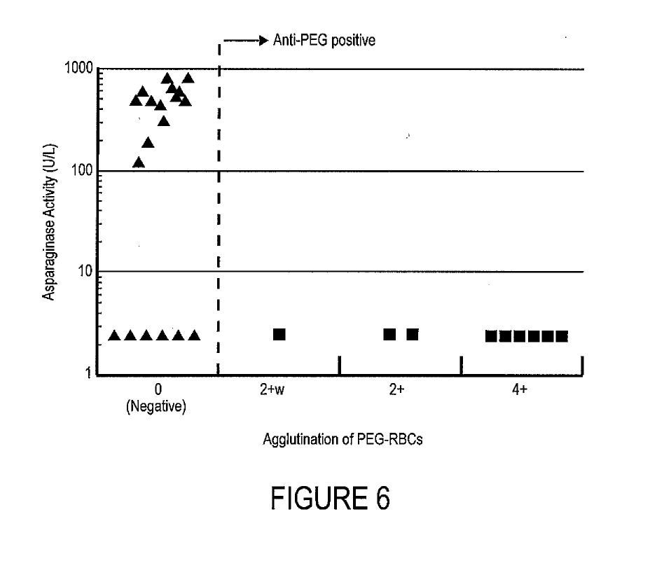

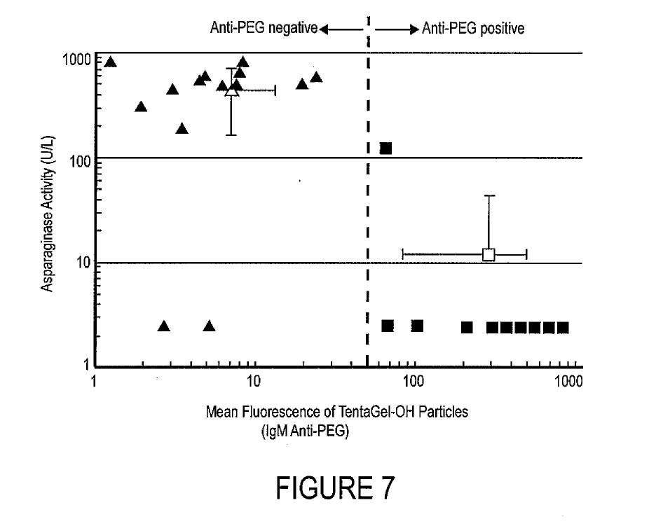

Regardless of which technique was used to determine anti-PEG (i.e., serology or flow cytometry), all PEG-ASNase-treated patient sera that were anti-PEG positive showed low or undetectable ASNase activity (FIGS. 6 and 7). Only one anomalous result was observed: one sample was weakly positive for anti-PEG IgM (mean fluorescence of 65) and also positive for anti-ASNase, but had ASNase activity at the low end of the therapeutic range (123 U/L).

### Conclusion:

The presence of anti-PEG is very closely associated with rapid clearance of PEG-ASNase for the samples analyzed in this study.

Many modifications and variation of the invention as hereinbefore set forth can be made without departing from the spirit and scope thereof and therefore only such limitations should be imposed as are indicated by the appended claims.

All patent and literature references cited in the present specification are hereby incorporated by reference in their entirety.

What is claimed is:

1. A method to reduce or prevent immune reactions against a PEG-conjugated therapeutic agent in a patient receiving said PEG-conjugated therapeutic agent, comprising:
selectively removing anti-PEG antibodies from the patient; and
administering said PEG-conjugated therapeutic agent to the patient, wherein removal of the anti-PEG antibodies prevents immune reactions in the patient and improves the therapeutic effectiveness of said PEG-conjugated therapeutic agent.

2. The method of claim 1, wherein said antibody removing step comprises performing an apheresis procedure in which the patient's blood is drawn and directed through an extracorporeal circuit having an anti-PEG antibody removing device containing a plurality of antibody discriminating elements for binding and sequestering said anti-PEG antibodies.

3. The method of claim 2, wherein said anti-PEG antibody removing device is an immunoadsorbent column having a solid substrate and a plurality of antibody discriminating elements immobilized thereon.

4. The method of claim 3, wherein said antibody discriminating elements comprise polymer molecules containing a sequence of at least 5 consecutive alkylene oxide repeat units, wherein said sequence represents the antigen required to form an antigen-antibody complex with an anti-poly(ethylene glycol) antibody.

5. The method of claim 3, wherein said solid substrate and a plurality antibody discriminating elements comprise inert particles.

6. The method of claim 5, wherein said inert particles have a diameter from about 10 microns to 500 microns.

7. The method of claim 6, wherein said inert particles are comprised principally of poly(ethylene glycol).

8. The method of claim 7, wherein said inert particles are PEG-liposomes.

9. The method of claim 4, wherein the polymer molecules immobilized on the solid substrate are poly(ethylene glycol) molecules with a molecular mass from about 300 g/mol to about 500,000 g/mol.

10. The method of claim 4, wherein said polymer molecules immobilized on the solid substrate are poly(ethylene glycol) molecules with a molecular mass of about 5,000 to about 20,000 g/mol.

11. The method of claim 4, wherein said polymer molecules are covalently bonded to the solid substrate.

12. The method of claim 4, wherein said sequence of at least 5 consecutive alkylene oxide repeat units is provided by poly(ethylene glycol) blocks in a block copolymer.

13. The method of claim 4, wherein said sequence of at least 5 consecutive alkylene oxide repeat units is provided methoxy-poly(ethylene glycol) molecules.

14. The method of claim 1 wherein said therapeutic agent is selected from the group consisting of: asparaginase, insulin, adenosine deaminase, arginine deaminase, glutaminase, camptothecin, filgrastin, recombinant human growth hormone, interferon, uricase, anti-tumor necrosis factor, anti-vascular endothelial growth factor, anti-granulocyte-stimulating factor, anti-granulocyte-macrophage colony stimulating factor, erythropoietin, thrombopoietin, anti-glutamic acid decarboxylase, anti-bacillus anthracis exotoxin, photosensitizer immunoconjugates, benzoporphyrin derivative-anti-epidemial growth factor receptor, superoxide dismutase, glucocerebrosidase, hemoglobin, and liposomes.

15. A method for preventing immune reactions in patients receiving PEG-conjugated therapeutic agents, comprising:
administering an anti-PEG antibody inactivating agent to the patient; and
administering the PEG-conjugated therapeutic agent to the patient.

16. The method of claim 15, wherein said anti-PEG antibody inactivating agent comprises at least one anti-PEG antibody discriminating element capable of forming antibody-antigen complex with the anti-PEG antibodies reactive against the PEG-conjugated therapeutic agent.

17. The method of claim 16, wherein said anti-PEG antibody discriminating element is a polymer molecule containing a sequence of at least 5 consecutive alkylene oxide repeat units, wherein said sequence represents the antigen required to form an antigen-antibody complex with an anti-poly(ethylene glycol) antibody.

18. The method of claim 17, wherein said polymer molecule is a poly(ethylene glycol) molecule with a molecular mass from about 300 g/mol to about 8,000,000 g/mol.

19. The method of claim 17, wherein said polymer molecule is a poly(ethylene glycol) molecule with a molecular mass of about 1,000 to about 20,000 g/mol.

20. The method of claim 17, wherein said polymer molecule is a poly(ethylene glycol) molecule with a molecular mass of about 20,000 to about 50,000 g/mol.

21. The method of claim 17, wherein said polymer molecule is poly(ethylene glycol) molecule covalently bonded to a polymeric core molecule selected from the group protein, divinylbenzene, polyethylenimine, polysaccharide.

22. The polymer molecule of claim 21, wherein said poly(ethylene glycol) molecules have a molecular mass of about 300 to about 50,000 g/mol.

23. The polymer molecule of claim 21, wherein said poly(ethylene glycol) molecules have a molecular mass of about 300 to about 1,000 g/mol.

24. The method of claim 17, wherein said sequence of at least 5 consecutive alkylene oxide repeat units is provided by poly(ethylene glycol) blocks in a block copolymer.

25. The method of claim 17, wherein said sequence of at least 5 consecutive alkylene oxide repeat units is provided by methoxy-poly(ethylene glycol) molecules.

26. The method of claim 15 wherein said therapeutic agent is selected from the group consisting of: asparaginase, insulin, adenosine deaminase, arginine deaminase, glutaminase, camptothecin, filgrastin, recombinant human growth hormone, interferon, uricase, anti-tumor necrosis factor, anti-vascular endothelial growth factor, anti-granulocyte-stimulating factor, anti-granulocyte-macrophage colony stimulating factor, erythropoietin, thrombopoietin, anti-glutamic acid decarboxylase, anti-bacillus anthracis exotoxin, photosensitizer immunoconjugates, benzoporphyrin derivative-anti-epidemial growth factor receptor, superoxide dismutase, glucocerebrosidase, hemoglobin, and liposome.

27. A device for removing from a patient anti-PEG antibodies reactive against a PEG-conjugated therapeutic agent, comprising:
a body having a fluid inlet, a fluid outlet, and a fluid passageway extending from the fluid inlet to the fluid outlet; and
a plurality of antibody discriminating elements contained in the fluid passageway that are capable of recognizing and sequestering anti-PEG antibodies,
wherein when the patient's blood is passed through the fluid passageway, the amount of anti-PEG antibody in the blood exiting the fluid outlet is lower than in the blood entering the fluid inlet.

28. A system for removing anti-PEG antibodies from a patient prior to treating the patient with a PEG-conjugated therapeutic agent, comprising:
an extracorporeal circuit for withdrawing blood from the patient, and then returning the processed blood back to the patient; and
an anti-PEG antibody removing device according to claim 27 operatively connected to said extracorporeal circuit.
